(19) United States
(12) Patent Application Publication
(10) Pub. No.: US 2023/0201292 A1
(43) Pub. Date: Jun. 29, 2023

(54) HERBAL FORMULATION FOR THE PREVENTION AND MANAGEMENT OF TYPE-2 DIABETES MELLITUS AND ASSOCIATED MICROVASCULAR COMPLICATIONS

(71) Applicants: Khushbu Kumari, Hisar (Haryana) (IN); Guru Prakash, Hisar (Haryana) (IN)

(72) Inventors: Govind Prasad Dubey, Varanasi (IN); Naveen Agrawal, Hisar (Haryana) (IN); Parul Agrawal, Hisar (Haryana) (IN); Kunka Mohanram Ramkumar, Tamil Nadu (IN); Rajappan Chandra Satishkumar, Tamil Nadu (IN); Ram Gopal Saini, Bathinda (IN); Rajesh Dubey, Hisar (Haryana) (IN)

(73) Assignees: Khushbu Kumari, Hisar (Haryana) (IN); Guru Prakash, Hisar (Haryana) (IN)

(21) Appl. No.: 17/692,899

(22) Filed: Mar. 11, 2022

(30) Foreign Application Priority Data
Dec. 28, 2021 (IN) 202111061100

(51) Int. Cl. A61K 36/37; A61K 45/06; A61P 3/10

(52) U.S. Cl. CPC A61K 36/37; A61K 45/06; A61P 3/10; A61K 2236/333

(57) ABSTRACT

Disclosed is an herbal formulation for the prevention and management of Type 2 diabetes and associated risk factors containing an effective amount of a hydro-alcoholic extract of Salacia roxburghii and an hydro-alcoholic extract of Salacia oblonga. Also disclosed are methods of preparing the formulation and using the formulation for the prevention and management of Type 2 diabetes and associated risk factors.

This application claims priority benefit from Indian Patent Application No. 202111061100, filed on Dec. 28, 2021, the entire content of which is incorporated herein by reference. All references cited anywhere in this specification are incorporated by reference as if each had been individually incorporated.

### FIELD OF INVENTION

The present invention relates to a novel herbal formulation and to the process thereof for the prevention and management of Type 2 diabetes mellitus and associated risk factors of coronary heart disease. The preparation of present invention is advantageous if used for the prevention and management of hyperglycemia, hyperhomocysteinemia, altered adipokines, elevated inflammatory cytokines, abnormal metabolism involved with diabetic patients. The beneficial role of novel herbal formulation can be assessed on blood glucose level, percent inhibition of alpha glucosidase, amylase and lipid lipase lowering activity, inflammatory pro-cytokines and adipokines as well as PPAR-α agonist activity.

### BACKGROUND OF INVENTION

The prevalence of diabetes (diabetes mellitus or DM) keeps on rising all over world; in particular Type 2 (or Type II) diabetes occurring in the adult population is dramatically increasing. Before 1990 the Type 2 diabetes was occurring after the age of forty years but from the year 2000 onwards Type 2 diabetes is diagnosed even among adolescents. The increasing number of diabetic from the younger age will definitely extend the health and economic problem not only to the individual and family but also to the community and nation. Earlier, genetics was the main cause. If parents or grandparents were diabetic, their children may develop diabetes. Now environmental factors play the dominant role.

The prevalence of Type 2 diabetes is expected to rise more rapidly in future. In 2000, the prevalence of Type 2 diabetes was estimated to be 0.19% in people<20 years old and 8.6% in people>20 years old. In individuals>65 years the prevalence of Type 2 diabetes was 20.1%. Globally, the number of people with diabetes is expected to rise from the current estimate of 150 million to 220 million in 2010 and 300 million in 2025. Now Type 2 diabetes has become one of the world's most important public health problems. Another study group estimated that there were 124 million persons with diabetes in the world in 1997 and predicted this number would grow to 221 million in 2010. The largest increase in the prevalence number is thought likely to appear in India, China and other developing countries.

Sex, age and ethnic background are important factors in determining risk for the development of Type 2 diabetes. The risk of Type 2 diabetes increases with age. This disorder is more common in female and increasing sharply with age. Other relevant risk factors are family history of diabetes, low birth weight, Gestational diabetes, high blood pressure, smoking, abundant alcohol consumption and unsatisfactory dietary patterns. The increasing prevalence of Type 2 diabetes is a global health problem and related to increasing prevalence of obesity due to western lifestyle. Research shows that Type 2 diabetes causes obesity. About 55% patients of Type 2 diabetes mellitus are obese, with the result obesity leads to increased insulin resistance that can develop into diabetes. Further, Type 2 diabetes mellitus often associated with obesity, hypertension and dyslipidemia. Additional factors found to be associated with Type 2 diabetes include ageing, high fat diets and less active lifestyle.

Increased awareness about diabetes is due to increased level of literacy, Media like print, and television also contribute for more number of cases. Improved detection rate is also on the increase because of the advent of blood sugar meters which makes it easier for anyone to diagnose diabetes anywhere on the earth.

The impact of diabetes is apparent from the data in India. India had only 2-3% of its population as diabetic in 1970, while in 2000, it was 14%. By 2025, almost 25% of Indian population will have diabetes. If 25% of Indian population is going to become Type 2 diabetic within 10-15 yrs., the remaining 75% of the population should take care of them. Otherwise, the entire Indian population will have to bear the burden of diabetes care—i.e., if one is getting admitted for diabetes problem one person should stay at hospital as an attender, another person has to be there in the house to prepare food etc. while another person should travel between the house and hospital. This will lead to enormous loss of man-hours and thus, inflicting heavy loss on the economy.

Postprandial hyperglycemia plays an important role in the development of Type 2 diabetes and cardiovascular complications, and has been proposed as an independent risk factor for cardiovascular diseases. The present test formulation is traditionally used in the prevention and treatment of diabetes. This formulation extract with water improves the intestinal and perivascular fibrosis in the hearts and it also reduced plasma glucose levels, whereas it has little effect in the fasted animals, suggesting inhibition of post prandial hyperglycemia in Type 2 diabetic animals, which might play a role in improvement of the cardiac complications.

Type 2 diabetes mellitus (sometimes abbreviated as T2DM) represents a global public health concern with rapidly increasing prevalence world-wide. It is a chronic metabolic disease characterized by a loss of insulin sensitivity by many cell types and progressive pancreatic j-cell dysfunction as a consequence of a hyperglycemia. It has long been appreciated that chronic activation of the innate immune system is associated with Type 2 diabetes mellitus. Growing evidence support the involvement of inflammatory processes with an abnormal production of cytokines and activation of inflammatory signalling pathways in the development of this metabolic disease. Despite underlying chronic inflammation, high incidence of community-acquired and nosocomial infectious diseases in Type 2 diabetes may indicate dysfunctions in innate immune response in these patients. Indeed, studies in Type 2 diabetes subjects have revealed impaired phagocytic, chemotactic activity of monocytes and neutrophils, decreased natural killer cell and dendrite cell function, and so on.

Oxidation and other forms of modification of lipids and lipoproteins have emerged as a major pathogenic factor in atherosclerosis. Modified lipoproteins deliver pro-inflammatory signals that activate innate and adaptive immune responses and disturb the integrity of the microvasculature. In diabetes, the combination of hyperglycemia and increased oxidative stress results in enhanced LDL modification. Advanced glycation end-products (AGE)-modified LDL plays an important pathogenic role through its interactions with RAGE and angeiotensin receptors. Oxidized LDL activates T cells, leading to enhanced inflammation through the release of macrophage-activating mediators. The adaptive humoral autoimmune response to modified forms of LDL is well characterized and strong evidence exists linking the formation of immune complexes (IC) involving modified forms of LDL and the corresponding auto-antibodies with the development of diabetic complications. High levels of oxidized LDL in IC strongly predict the progression of atherosclerosis in patients with type-1 diabetes, while high levels of malondialdehyde-modified LDL in IC indicate strong risk for acute cardiovascular events in patients with Type 2 diabetes.

Monocytes are the main cells of innate immunity that play central role in the initiation and resolution receptor TLR4 results in production of a broad of inflammatory response to pathogens. Recognition of LPS by its range of molecules, including proinflammatory cytokines, chemokines, reactive oxygen species, and up-regulation of adhesion and co-stimulatory receptor. At the same time up regulation of anti-inflammatory mediators ensures successful resolution of the inflammatory response. A delicate balance between pro and anti-inflammatory responses is required to prevent the progression from no resolving acute inflammation to persistent chronic inflammation. Although coenocytes are the main calla producing pro-inflammatory cytokines in norm, controversial result have been obtained concerning their potential role in inflammatory processes.

These biochemical factors are elevated inflammatory markers like IL-6, TNF-α and hs-CRP. Since plasma homocysteine level can be reduced by folic acid and vitamin B6, it seems possible that intake of these vitamins may provide protection against diabetes. In case of childhood obesity and familial hypercholesterolemia these changes are apparent in comparison to normal population. Moreover, plant foods rich in fibre, antioxidant, vitamins, minerals, unsaturated fatty acids, folic acids, phytoestrogen and phenolic compounds may also have a potential preventive effect against development of diabetes due to their antioxidant property.

### SUMMARY OF INVENTION

The present disclosure is focused on identifying individuals at risk of development of diabetes and inflammation.

Keeping the above facts in view the present invention provides a plant-based formulation showing beneficial role in the management of Type 2 diabetes mellitus and associated CHD risk factors. In Ayurvedic system of medicine, several plants have been advocated for their hypoglycemic effects and are still in practice. Taking the lead from ancient literature two plants Salacia roxburghii and Salacia oblonga were selected out of various screened plants done for this purpose and the novel formulation was prepared following standard norms.

In experimental studies the safety and efficacy profile of combined formulation has been established. In pre-clinical trials the test formulation exerted α-glucosidase inhibitory activity. The test drug was found to have an agonist for PPAR-α activated receptor and regulates insulin responsive gene transcription involved in glucose production, transport and utilization and thus reduced blood glucose and reduces hyperinsulinemia. Our claims are also established on the basis of experimental and clinical trials.

An object of the present invention is to provide a herbal formulation beneficial in the management of Type 2 diabetes mellitus and associated CHD risk factors with the object to prevent the CHD death among diabetic population.

Another object of present invention is to provide a herbal formulation having α-glucosidase inhibitory activity and can act as prescription drug “Acarbose”.

Still, another object of present invention is to provide an herbal formulation showing beneficial role on lipid metabolism (dyslipidemia) among Type 2 diabetes cases.

Further, object of present invention is to provide a herbal formulation showing role as an agonist for PPAR activated receptor and can regulate insulin responsible gene transcription.

Yet further object of present invention is to provide a herbal formulation which can delay the pro-inflammatory markers TNF-α, and hsCRP in Type 2 diabetes cases.

Further, object of present invention is to provide a herbal formulation effective in lowering the, elevated homocysteine level in diabetes patients.

Yet another object of present invention is to provide a herbal formulation showing beneficial role in reducing leptin and increase adiponectin among Type 2 diabetes cases to prove the anti-obesity property of present novel formulation.

According to this invention there is provided a novel herbal formulation for the prevention and management of Type 2 diabetes mellitus and associated microvascular complication like obesity, dyslipidemia, vascular inflammation, atherosclerosis and oxidative injury. The formulation comprising of at least one plant or preferably two plants i.e. Salacia roxburghii and Salacia oblonga.

Further, according to this invention there is provided a process for the preparation of present novel plant based formulation by preparing hydro-alcoholic extract of Salacia roxburghii (root) and Salacia oblonga (root) by using water (aqueous) and ethanol (30:70) at 70-80° C. and maintaining pH of solution between 7-10, separating chromatographically the active compound by using TLC, HPLC and HPTLC, supported with molecular characterization of the plant extract by using IR and NMR.

The present invention is directed to an herbal formulation for the prevention and management of Type 2 diabetes and associated risk factors containing an effective amount of a hydro-alcoholic extract of Salacia roxburghii and a hydro-alcoholic extract of Salacia oblonga. The herbal formulation can also include one or more of minerals, vitamins, salts, filler and binders. In exemplary embodiments, the formulation contains an amount to provide a dosage of Salacia roxburghii of 450-900 mg/day, and a dosage of Salacia oblonga of 400-900 mg/day. In exemplary embodiments, the hydro-alcoholic extracts are extracts of Salacia roxburghii root and Salacia oblonga root.

The present invention also includes a method of managing Type 2 diabetes and associated CHD risk factors in a subject suffering from diabetes by administering the herbal formulation of claim 1 to the subject. The formulation and its administration can provide a number of beneficial biological effects including one or more of: providing alpha glucosidase, amylase inhibitory properties and lipid lipase lowering effect; the extract of Salacia roxburghii acting as an agonist for peroxisome proliferaters activators (PPAR) activated receptor γ and α, and regulates insulin responsiveness gene transcription involved in glucose production transport and utilization, thus reducing blood glucose level; the hydro-alcoholic extract of Salacia roxburghii providing potent anti-diabetic role; reducing elevated triglycerides along with blood glucose level; reducing Apolipo B and increases good cholesterol HDL-c; providing an anti-atherosclerotic property by reducing oxidized LDL-c in diabetes and improving endothelial dysfunction; reducing vascular inflammation by reducing IL-6, TNF-α and CRP; and providing an anti-atherosclerotic and anti-obesity effects providing antioxidant potentials through homocysteine regulation.

The present invention is also a method for preparing the herbal formulation by preparing a hydro-alcoholic extract of Salacia roxburghii and a hydro-alcoholic extract Salacia oblonga by extracting the Salacia roxburghii and Salacia oblonga with aqueous ethanol in 30:70 ratio of water to ethanol at 70-80° C. while maintaining pH of solution between 7-10; subjecting the hydro-alcoholic extracts to separation by chromatography, and characterizing molecules in the extracts by using IR and NMR. In exemplary embodiments, the roots pf the plants are extracted.

### BRIEF DESCRIPTION OF THE FIGURES

FIG. 1 is a flow chart showing extraction steps of the invention,

FIG. 2 shows the beneficial role of the herbal formulation on fasting blood glucose level among Type 2 Diabetes Mellitus cases,

FIG. 3 shows the decrease in post prandial blood glucose level with the herbal formulation in the case of Type 2 Diabetes Mellitus cases,

FIG. 4 shows the decrease in Interleukin 6 with the herbal formulation in Type 2 Diabetes Mellitus cases,

FIG. 5 depicts the effect of herbal composition on CRP level in of Type 2 Diabetes Mellitus cases,

FIG. 6 depicts the decrease in inflammatory biomarker following herbal formulation in treatment of Type 2 Diabetes Mellitus cases,

FIG. 7 shows the beneficial role of the herbal formulation on endothelin level among Type 2 Diabetes Mellitus cases,

FIG. 8 shows decrease in Apolipoprotein B following herbal formulation treatment in Type 2 Diabetes Mellitus cases,

FIG. 9 depicts the beneficial role of the herbal formulation in decreasing the Homocysteine level among Type 2 Diabetes Mellitus cases,

FIG. 10 shows the decrease in the Leptin level after the treatment of herbal drug formulation in Type 2 Diabetes Mellitus cases,

FIG. 11 shows the modulation of adiponectin after the administration of the herbal formulation in Type 2 Diabetes Mellitus cases.

### DETAILED DESCRIPTION OF THE INVENTION

The hydro-alcoholic extract of two Ayurvedic plants Salacia roxburghii and Salacia oblonga by using 30:70 ratio of water and ethanol respectively is used for the experimental and clinical studies. The water utilized for extraction was decontaminated for any type of bacterial or abnormal growth by using reverse osmosis plant. After extraction the active molecules was identified and separated by HPLC, HPTLC and NMR procedures.

The biological activity was studied on the basis of mode of action of the test drug and effect of the drug on various parameters undertaken for this clinical condition. The molecular characterization was done by using NMR and bio-molecular reaction following the interaction between the chemical and biological markers like insulin resistance (blood glucose levels), inflammatory cytokines, adipokines, various fractions of lipids including triglycerides, homocysteine etc.

The pre-clinical toxicological studies were carried out to determine the safety profile of individual plant candidate as well as all two candidates combined to prepare novel formulation before using the drug for human beings. The mode of action of single plant and combined formulation was carried out in animal models.

The beneficial role of test formulation on fasting and postprandial blood glucose level, α-glucosidase inhibitory activity, PPAR-α agonist activity, insulin resistance, abnormal lipids, atherosclerosis, altered adipokines and inflammatory cytokines etc. were established in animal models before using the drug for human consumption.

### Extraction Procedure:

The shed dried root of Salacia roxburghii and Salacia oblonga were utilized for obtaining extracted material of the plants. The water and ethanol extract (30:70 ratio) were utilized for the extraction of active compound found in the plants. After extraction the extracted materials were taken for chromatographic separation by using TLC, HPLC, and HPTCL. After identification and separation of the active compound the molecular characterization was carried out by using IR and NMR.

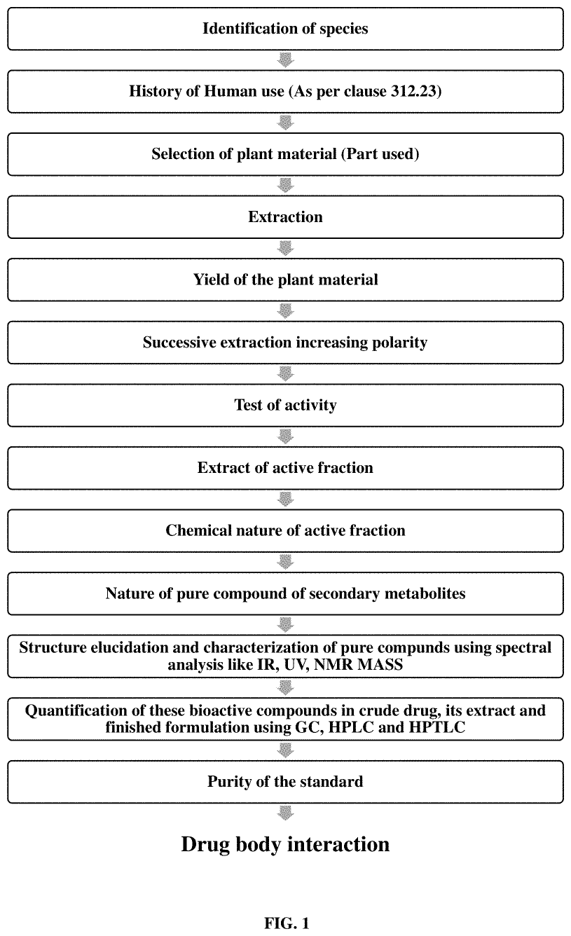

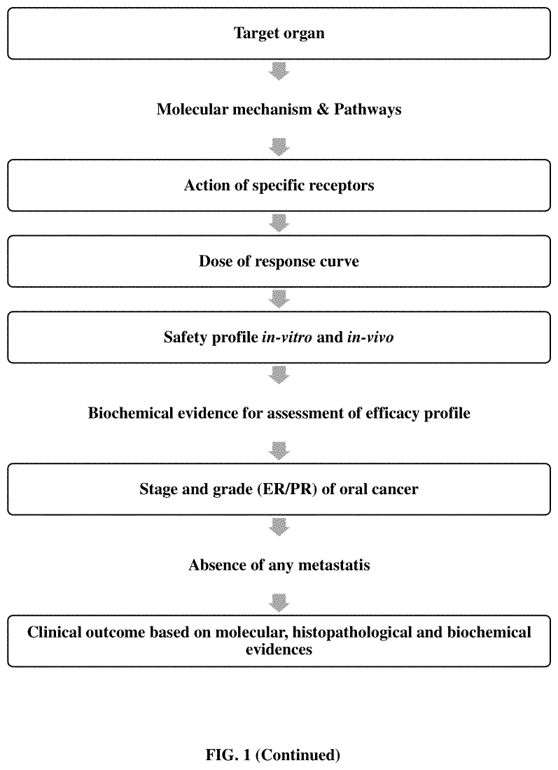

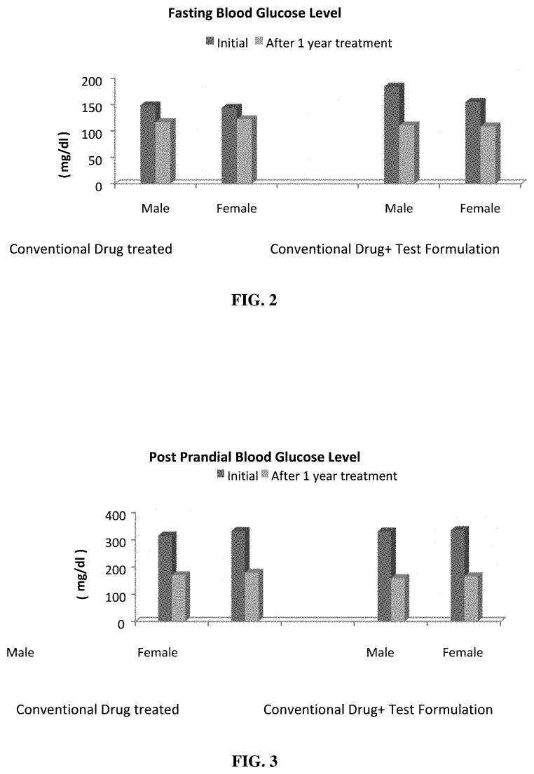

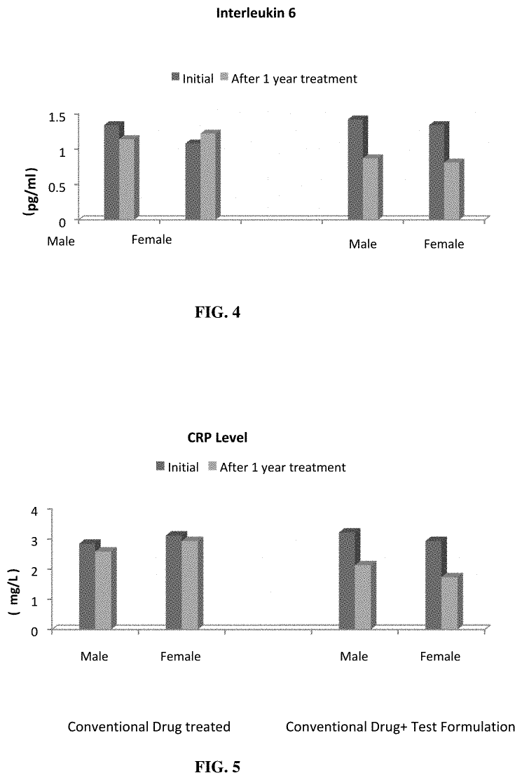

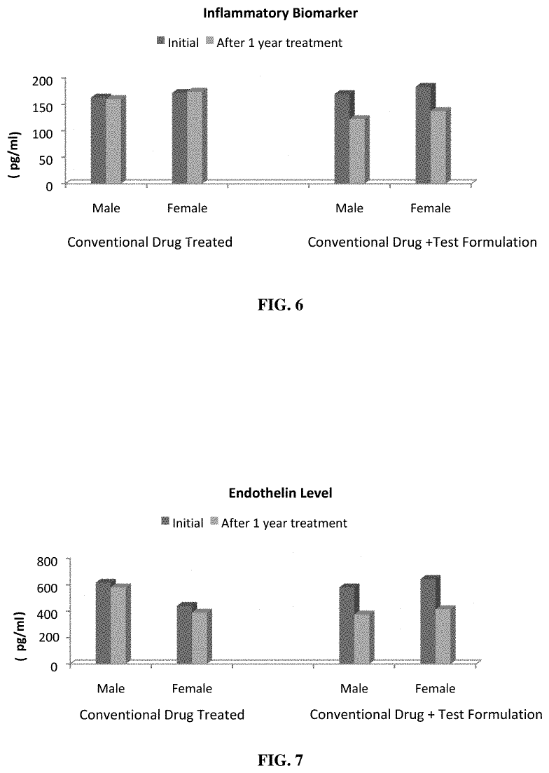

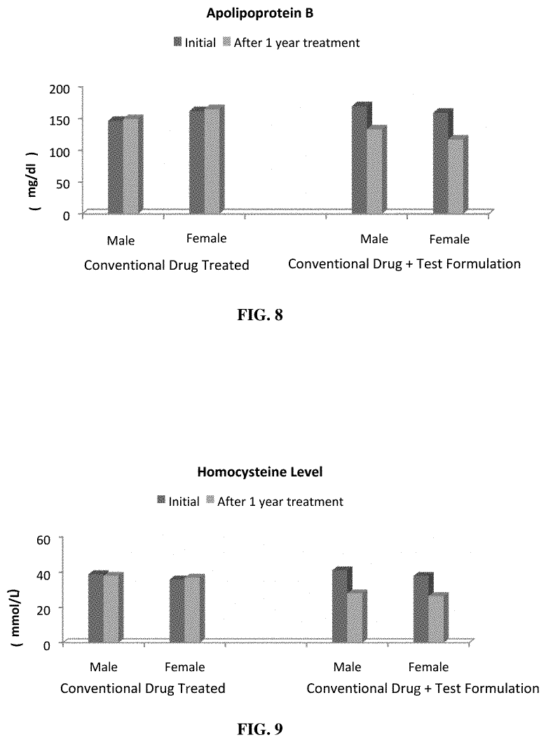

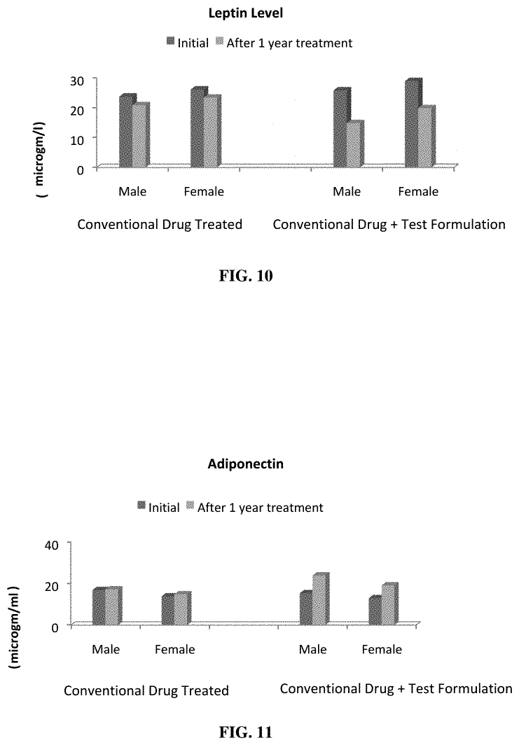

The extraction was done at the temperature of 70-80C. The pH of the solution was maintained between 7-10. FIG. 1 shows the steps followed and carried out to isolate the active compound and preparation of test drug.

According to this invention there is provided an Ayurvedic formulation for the prevention and management of Type 2 diabetes and associated cardiac risk factors with the object to prevent the diabetic patients from cardiac death and also from various type of morbidity caused due to impaired glucose tolerance. The present test formulation comprising of the following two ingredients—

1. Salacia roxburghii - Root
2. Salacia oblonga - Root

Preferably the aforesaid plants are present in the test drug in following doses—

| Name of the plants | Dose |
|---|---|
| 1. Salacia roxburghii | 450-900 mg/day |
| 2. Salacia oblonga | 400-900 mg/day |

The formulation may also comprise known additives such as minerals, vitamins, salts, filler (for capsulation or tablet) and binders, if required to present in trace amount.

Thus any known additive or supplement is added to prepare the final formulation as required and present in trace amount. Reference is made here in capsule form. However, it would be apparent that the preparation may also be in the form of tablet.

Preferably but without implying any limitation the preparation comprises—

| Name of the plants | Dose |
|---|---|
| 1. Salacia roxburghii | 450 mg/day |
| 2. Salacia oblonga | 450 mg/day |

The present herbal formulation is based on the combined effect of two plant extract namely Salacia roxburghii and Salacia oblonga. This novel formulation has shown α-glucosidase inhibitory activity, PPAR agonist activity, modified abnormal lipids including triglyceride, reduced inflammatory cytokines, regulates adipokines, improves cardiac functions. Those effect are mediated through alpha-glucosidase and amylase inhibition including reduction in lipid lipase concentration, PPAR agonist activity that results in insulin responsive gene transcription (involved in glucose production, transport and utilization) ultimately reduces blood glucose level, reduces inflammatory process by reducing CRP, IL-6, TNF-α, it regulates abnormal lipids including triglycerides concentration, decreases leptin level and enhances adiponectin. All these beneficial effects ultimately slowed down/improved the atherosclerotic process among diabetes patients and has potential role in prevention of adverse cardiac event.

Salacia roots modulate multiple targets like peroxisome proliferator-activated receptor-alpha-mediated lipogenic gene transcription, angiotensin II/angiotensin II type 1 receptor, alpha-glucosidase, aldose reductase and pancreatic lipase. These multi-target actions may mainly contribute to Salacia root-induced improvement of Type 2 diabetes and obesity-associated hyperglycemia, dyslipidemia and related cardiovascular complications seen in humans and rodents. The results of bioassay-guided identification indicate that mangiferin, salacinol, kotalanol and kotalagenin 16-acetate are at least in part responsible for these multi-target regulatory activities of Salacia roots. The evidence suggests that this unique traditional medicine fulfils a multiple-target strategy in the prevention and treatment of diabetes and obesity. Although toxicological studies have suggested minimal adverse effects of the herbal medicine in rodents, a clinical trial is crucial to further confirm the safety of Salacia roots. In addition, further mechanistic studies are necessary in order to allow a better understanding of how use of Salacia root may interact with other therapeutic interventions. Salacia oblonga containing various active components have been found to meet multiple targets in diabetes and obesity through modulating PPAR-α mediated lipogenic gene transcription and AT1 signalling and inhibiting α-glucosidase

As diabetes increases the risk of myocardial infarction and risk of stroke, therefore the CHD risk factors particularly lipid abnormalities must be treated aggressively to reduce the risk of atherosclerosis. Further, it is reported that α-glucosidase inhibitors do not increase insulin level and their main effect is to lower postprandial blood glucose, prevent from weight gain. It is hypothesized that present test formulation improves the glycemic load by improving insulin sensitivity, as a PPAR-α agonist, stimulates beta-cells, increases glucose uptake in tissues, may have activity on cathepsin and increases cyclic AMP count on islets. As synergism test drug has antioxidant effects.

In the present study, treatment with test formulation significantly reduced the level of homocysteine indicating increased endothelium dependent flow-mediated dilation in patient with Type 2 diabetes mellitus. Atherosclerosis occurs due to a number of factors in diabetic individuals. Both insulin resistance and elevated lipid levels are the triggering factors for atherogenic injury, endothelial dysfunction in diabetics otherwise is more prone to atherogenic injury due to decreased production of endothelial nitric oxide and increased production of plasminogen activator inhibitor. Lipid disorders like elevated total cholesterol, HDL-c and triglyceride is the major risk factor for CHD. Therefore, the prevention and management of risk factors of cardiovascular is the basic and essential principle among diabetes patients to prevent them from cardiac arrest.

### About the Plants:

1. Salacia roxburghii: Mainly found in Eastern part of India. Salacia species belongs to family Hippocrastaceae. Fruits and root are used for medicinal purpose. Salaretin is the main active compound isolated from this plant is responsible for breakdown of starch in diet. Mangiferin is also an important component of Salacia roxburghii. This drug has shown anti-diabetic, anti-inflammatory, anti-oxidant, anti-obesity and homocysteine lowering activity in diabetes patients. Further, the drug enhanced PPAR-α—mediated lipogenic gene expression. Mangiferin lowers blood lipids in diabetes.
2. Salacia oblonga is a member of the Hippocrastaceae family commonly known as “saptarangi” due to its golden colored root bark. It is widely distributed in south region of India and Sri Lanka. Salacia oblonga is a perennial wild, woody, climbing plant found in foot hills of the mountain of Eastern and Western Ghats of Tamilnadu and Kerla. The seed of Salacia oblonga is light brown and orange in colour and its fruits are globose and tuberculata. In Ayurvedic system of Medicine it has been used as a treatment of diabetes. This is due to salacia's “mangiferin” component, which inhibits sugar absorption and also increases the body's sensitivity to insulin. The roots and stems of Salacia oblonga have been used as a treatment for diabetes in the ayurvedic system of medicine. A highly potent α-glucosidase inhibitor, kotalanol 1, has now been isolated from this plant. One of the conventional therapeutic approaches to diabetes management is through the use of alpha-glucosidase inhibitors that lower glucose levels by blocking the enzymes that digest starches in the intestines.

### EXAMPLES

### Example-I

When the hydroalcoholic extract of Salacia roxburghii (100 mg/kg) was given to Sprague Dawley rats 46 percent α-glucosidase inhibitory activity was recorded. Further, amylase and lipid lipase lowering effects were also noticed.

### Example-II

When the hydro-alcoholic extract of Salacia roxburghii (100 mg/kg) was given to streptozotocin induced diabetic rats a significant reduction in blood glucose level was measured indicating anti-diabetic role of the drug.

### Example-III

When the hydro-alcoholic extract of Salacia roxburghii in the dose of 100 mg/kg was given to streptozotocin induced diabetic rats, the drugs exerted agonist for PPAR activated receptors resulting in insulin regulated gene transcription.

### Example-IV

When the organic extract of Salacia roxburghii in the dose of 900 mg/day (in two divided doses) was given to diagnosed cases of Type 2 diabetes a significant decrease in postprandial blood glucose level was noticed. An average 40 percent depletion of blood glucose level indicated the anti-diabetic activity of test drug.

### Example-V

When the hydro-alcoholic extract of Salacia roxburghii (475 mg/day) and Salacia oblonga (450 mg/day) was mixed and given to Type 2 diabetes patients a marked decrease in triglycerides content was estimated along with blood glucose lowering property of the test formulation.

### Example-VI

When the hydro-alcoholic extract of Salacia oblonga (525 mg/day) and Salacia roxburghii (425 mg/day) mixed and administered to Type 2 diabetes patients a significant decrease in Apolipo-B and moderate increase in HDL-c level was noticed.

### Example-VII

When the hydro-alcoholic extract of Salacia oblonga (550 mg/day) and Salacia roxburghii (350 mg/day) mixed and given to diagnosed cases of Type 2 diabetes subjects the oxidized LDL-c level decreased indicating improvement in atherosclerotic process among Type 2 diabetes patients.

### Example-VIII

When the hydro-alcoholic extract of Salacia roxburghii (575 mg/day) and Salacia oblonga (350 mg/day) was mixed and given to diabetes patients decrease in proinflammatory markers like IL-6, TNF-α including hs.CRP was estimated indicating improvement in vascular inflammation in diabetes cases.

### Example-IX

When the hydro-alcoholic extract of Salacia roxburghii (450 mg/day) and Salacia oblonga (450 mg/day) was mixed and administered to selected diabetes patients increase in adiponectin and decrease in leptin level indicated the anti-atherosclerotic and antiobesity role of test formulation.

### Example-X

When the hydro-alcoholic extract of Salacia roxburghii (625 mg/day) and Salacia oblonga (325 mg/day) mixed and given to diabetes patients the elevated homocysteine level markedly decreased following treatment. This study indicated the anti-oxidant activity of this combined drug.

### Example-XI

When the organic extract of Salacia roxburghii (625 mg/day) and Salacia oblonga (325 mg/day) mixed and orally administered to selected Type 2 diabetes patients a better effectivity of test formulation was recorded. The blood glucose level is decreased, abnormal lipids modified including apolipo-B, the inflammatory cytokines IL-6 and TNF-α decreased, CRP and leptin also reduced, where as adiponectin level increased and elevated Hcy decreased. This novel combined formulation has shown potential role in the prevention and management of cardiovascular risk factors among diabetic patients through regulation of PPAR-α and PPAR-7 and also due to its aglucosidase inhibitory activity resulting in control of glycemic index and enhancing insulin sensitivity. Thus it is proposed as a better safer remedial measure for the management of Type 2 diabetes mellitus patients.

It is to be noted that the present invention is susceptible to modifications, adaptations and changes by those skilled in the art. Such variant embodiments employing the concepts and features of this invention are intended to be within the scope of the present invention.

Alpha-glucosidase inhibitory activity of single
as well as combined formulation

| | Percent inhibition | | |
|---|---|---|---|
| Concentration | Salacia | Salacia | Combined |
| (μg/ml) | roxburghii | oblonga | formulation |
| 50 mg/kg | 25.88 | 22.36 | 29.75 |
| 100 mg/kg | 41.82 | 42.29 | 58.55 |
| 150 mg/kg | 44.6 | 32.21 | 63.94 |

### Experimental Study-I

TABLE I-2
Reduction in blood glucose level following test
drug treatment in Streptozotocin (STZ) treated diabetic rats

| | Blood glucose level (mg/d ) | | |
|---|---|---|---|
| Treated group | 7^{th} Days | 14^{th} Days | 28^{th} Days |
| Normal control (N = 6) * | 58.36 ± 8.24 | 57.25 ± 9.31 | 59.24 ± 11.27 |
| Td. With STZ (65 mg/kg) | 328.68 ± 26.97 | 285.60 ± 25.65 | 255.45 ± 34.69 |
| N = 6 ** | | | |
| Td. With STZ + Test drug | 247.49 ± 21.37 | 205.32 ± 24.43 | 165.80 ± 19.27 |
| (N = 6)*** | | | |
| Td. With STZ + | 228.33 ± 19.44 | 192.74 ± 15.18 | 157.51 ± 10.24 |
| Acarbose**** | | | |

Comp.
*vs** P < 0.001 P < 0.001 P < 0.001
**vs*** P < 0.001 P < 0.001 P < 0.001
***vs**** P > 0.05 P < 0.05 P < 0.05

TABLE I-2
Reduction in hs C-reactive protein following test
drug treatment in STZ treated diabetic rats

| | hs CRP (mg/L) | | |
|---|---|---|---|
| Treated group | 7^{th} Days | 14^{th} Days | 28^{th} Days |
| Normal control (N = 6) * | 1.45 ± 0.35 | 1.36 ± 0.28 | 1.25 ± 0.39 |
| Td. With STZ (65 mg/kg) | 8.26 ± 2.36 | 7.65 ± 2.08 | 5.36 ± 1.67 |
| N = 6 ** | | | |
| Td. With STZ + Test drug | 6.27 ± 1.57 | 5.27 ± 0.82 | 3.97 ± 0.72 |
| (N = 6)*** | | | |
| Td. With STZ + | 6.78 ± 1.52 | 5.43 ± 0.75 | 4.97 ± 0.87 |
| Acarbose**** | | | |

Comp.
*vs.** P < 0.001 P < 0.001 P < 0.001
**vs*** P < 0.001 P < 0.001 P < 0.001
***vs**** P > 0.05 P > 0.05 P > 0.05

TABLE I-3
Reduction in Interleukin-6 following test
drug treatment in STZ treated diabetic rats

| | IL-6 (pg/ml) | | |
|---|---|---|---|
| Treated group | 7^{th} Days | 14^{th} Days | 28^{th} Days |
| Normal control (N = 6) * | 0.79 ± 0.12 | 0.89 ± 0.12 | 0.82 ± 0.18 |
| Td. With STZ (65 mg/kg) | 4.05 ± 1.10 | 3.84 ± 0.97 | 3.27 ± 0.79 |
| N = 6 ** | | | |
| Td. With STZ + Test | 2.58 ± 0.27 | 2.12 ± 0.32 | 1.60 ± 0.24 |
| drug (N = 6)*** | | | |
| Td. With STZ + | 2.87 ± 0.25 | 2.49 ± 0.32 | 2.23 ± 0.26 |
| Acarbose**** | | | |

Comp.
*vs.** P < 0.001 P < 0.001 P < 0.001
**vs*** P < 0.001 P < 0.001 P < 0.001
***vs**** P > 0.05 P > 0.05 P > 0.05

TABLE I-4
Effect of Herbal formulation on adiponectin
level in STZ treated diabetic rats

| Treated group | Adiponectin (μg/ml) | | |
|---|---|---|---|
| | 7^{th} Days | 14^{th} Days | 28^{th} Days |
| Normal control (N = 6)* | 13.28 ± 1.90 | 14.36 ± 1.98 | 14.40 ± 2.08 |
| Td. With STZ | 6.79 ± 1.12 | 5.75 ± 0.98 | 5.11 ± 0.85 |
| (65 mg/kg) N = 6 ** | | | |
| Td. With STZ + Test | 9.27 ± 1.98 | 10.22 ± 1.69 | 12.63 ± 2.08 |
| drug (N = 6)*** | | | |
| Td. With | 8.87 ± 2.10 | 9.65 ± 1.63 | 10.23 ± 2.14 |
| STZ + Acarbose **** | | | |

TABLE II-3
Role of Herbal formulation on body weight
following cafeteria diet in experimental rats

| | | | | Comp. Initial |
|---|---|---|---|---|
| | Body Weight (grams) | | | vs After 30 |
| Treated group | Initial | After 15 Days | After 30 Days | days |
| Normal control | 104.48 ± 3.75 | 109.68 ± 6.22 | 116.25 ± 4.93 | P < 0.001 |
| (N = 6) | | | | |
| Cafeteria diet only | 98.28 ± 4.35 | 127.58 ± 6.14 | 156.83 ± 11.20 | P < 0.001 |
| (N = 6) | | | | |
| Cafeteria diet + test | 112.63 ± 9.43 | 118.26 ± 8.11 | 125.67 ± 7.52 | P < 0.001 |
| formulation (N = 6) | | | | |

### Experimental Study-II

TABLE-II-2
Effect of herbal formulation on total cholesterol and triglycerides
following cafeteria dies in experimental animals

| | TC (mg/dl) | | Comp. | TG (mg/dl) | | Comp. |
|---|---|---|---|---|---|---|
| | | After | Initial vs | | After | Initial vs |
| | | 30 | After 30 | | 30 | After 30 |
| Treated group | Initial | Days | days | Initial | Days | days |
| Normal control | 82.68 ± | 86.38 ± | P > 0.05 | 81.75 ± | 82.23 ± | P > 0.05 |
| (N = 6) | 4.36 | 5.27 | | 7.98 | 9.28 | |
| Cafeteria diet | 86.21 ± | 90.46 ± | P < 0.05 | 78.58 ± | 94.25 ± | P < 0.05 |
| only (N = 6) | 6.43 | 8.95 | | 5.73 | 4.65 | |
| Cafeteria diet + | 84.74 ± | 80.37 ± | P < 0.05 | 82.15 ± | 73.48 ± | P < 0.05 |
| test formulation | 7.98 | 5.93 | | 8.73 | 7.29 | |
| (N = 6) | | | | | | |

TABLE-II-3
Effect of Herbal formulation on blood glucose and adiponectin level
following cafeteria dies in experimental rats

| | Blood glucose | | Comp. | Adiponectin | | Comp. |
|---|---|---|---|---|---|---|
| | level (mg/dl) | | Initial vs | (pg/ml) | | Initial vs |
| Treated | | After 30 | After 30 | | After | After 30 |
| group | Initial | Days | days | Initial | 30 Days | days |
| Normal | 59.85 ± | 57.70 ± | P > 0.05 | 11.78 ± | 12.264 ± | P > 0.05 |
| control | 6.29 | 6.38 | | 1.92 | 2.16 | |
| (N = 6) | | | | | | |
| Cafeteria | 55.67 ± | 72.28 ± | P < 0.001 | 6.54 ± | 7.85 ± | P < 0.001 |
| diet only | 6.08 | 5.97 | | 0.97 | 1.10 | |
| (N = 6) | | | | | | |
| Cafeteria | 58.28 ± | 65.36 ± | P < 0.01 | 7.82 ± | 10.17 ± | P < 0.01 |
| diet + test | 6.27 | 4.85 | | 1.03 | 1.84 | |
| formulation | | | | | | |
| (N = 6) | | | | | | |

TABLE II-4
Role of Herbal formulation on Total Cholesterol
among high cholesterol diet treated rats

| | Total cholesterol level (mg/dl) | | |
|---|---|---|---|
| Treated group | Initial | After 15 Days | After 1 months |
| Normal control (N = 10)* | 65.42 ± 7.79 | 64.87 ± 6.48 | 65.65 ± 8.37 |
| High cholesterol diet | 654.31 ± 50.24 | 887.29 ± 48.37 | 482.49 ± 38.58 |
| (N = 10)** | | | |
| High cholesterol diet + | 567.97 ± 78.29 | 658.36 ± 72.48 | 405.28 ± 46.15 |
| (N = 10)*** | | | |
| test formulation | | | |
| High cholesterol diet + | 691.52 ± 78.85 | 691.52 ± 78.85 | 280.50 ± 16.80 |
| statin (2.5mg/kg/day) | | | |
| (N = 10)**** | | | |

Comp.
*vs** P > 0.05 P < 0.001 P < 0.001
**vs*** P < 0.001 P < 0.001 P < 0.001
***vs**** P < 0.001 P < 0.001 P < 0.001

TABLE II-5
Effect of Herbal formulation on HDL-c level among
high cholesterol diet treated rats

| | HDL-c level (mg/dl) | | |
|---|---|---|---|
| Treated group | Initial | After 15 Days | After 1 months |
| Normal control (N=10)* | 22.25 ± 4.30 | 23.12 ± 2.68 | 22.12 ± 3.49 |
| High cholesterol diet | — | 16.39 ± 4.85 | 12.97 ± 1.68 |
| (N = 10)** | | | |
| High cholesterol diet + | — | 17.58 ± 2.46 | 19.46 ± 2.72 |
| test formulation | | | |
| (N = 10)*** | | | |
| High cholesterol diet + | — | 20.24 ± 4.76 | 21.43 ± 3.25 |
| statin (2.5 mg/kg/day) | | | |
| (N = 10)**** | | | |

Comp.
*vs** P > 0.05 P < 0.05 P < 0.001
**vs*** P > 0.05 P < 0.001
***vs**** P > 0.05 P < 0.05

TABLE II-6
Effect of Herbal formulation on LDL-c level
among high cholesterol diet treated rats

| | LDL-c level (mg/dl) | | |
|---|---|---|---|
| Treated group | Initial | After 15 Days | After 1 months |
| Normal control | 23.74 ± 4.59 | 22.27 ± 5.12 | 23.75 ± 6.19 |
| (N = 10)* | | | |
| High cholesterol diet | 298.65 ± 62.34 | 328.17 ± 58.27 | 312.33 ± 46.27 |
| (N = 10)** | | | |
| High cholesterol diet + | 197.32 ± 34.18 | 265.18 ± 39.11 | 137.98 ± 29.10 |
| test formulation | | | |
| (N = 10)*** | | | |
| High cholesterol diet + | 168.34 ± 24.36 | 253.68 ± 36.45 | 107.78 ± 16.34 |
| statin (2.5 mg/kg/day) | | | |
| (N = 10)**** | | | |

Comp.
*vs** P > 0.05 P < 0.001 P < 0.001
**vs*** P < 0.05 P < 0.05 P < 0.001
***vs**** P < 0.05 P > 0.05 P < 0.05

TABLE II-7
Effect of Herbal formulation on Triglycerides level among high
cholesterol diet treated rats

| | Tr glycerides level (mg/dl) | | |
|---|---|---|---|
| Treated group | Initial | After 15 Days | After 1 months |
| Normal control (N = 10)* | 27.82 ± 8.65 | 30.64 ± 7.73 | 28.22 ± 5.47 |
| High cholesterol diet | 301.68 + 59.12 | 338.67 ± 63.75 | 297.12 ± 38.48 |
| (N = 10)** | | | |
| High cholesterol diet + | 201.65 ± 49.37 | 242.17 ± 59.35 | 187.90 ± 26.87 |
| test formulation | | | |
| (N = 10)*** | | | |
| High cholesterol diet + | 195.34 ± 19.67 | 227.65 ± 30.12 | 113.63 ± 18.84 |
| statin (2.5 mg/kg/day) | | | |
| (N = 10)**** | | | |

Comp.
*vs** P > 0.05 P < 0.001 P < 0.001
**vs*** P < 0.05 P < 0.05 P < 0.01
***vs**** P < 0.05 P > 0.05 P < 0.05

### Clinical Study

TABLE-CS-1
Effect of Herbal formulation on Body Mass Index among Type 2
diabetes mellitus cases

| | | | | | | Comp. |
|---|---|---|---|---|---|---|
| | | | Body Mass Index | | | Initial vs |
| | | No. of | | 6 Months | 1 Year | After 1 year |
| Treatment | sex | Cases | Initial | Treatment | Treatment | treatment |
| Conventional | M | 61 | 31.62 ± | 30.71 ± | 29.62 ± | P < 0.001 |
| Drug Treated | | | 3.01 | 3.04 | 3.01 | |
| | F | 38 | 29.26 ± | 28.97 ± | 28.04 ± | P < 0.001 |
| | | | 2.94 | 2.76 | 2.16 | |
| Conventional | M | 72 | 29.75 ± | 28.63 ± | 27.73 ± | P < 0.001 |
| Drug + | | | 2.89 | 2.95 | 2.90 | |
| Herbal | F | 43 | 30.26 ± | 29.63 ± | 27.68 ± | P < 0.001 |
| Formulation | | | 2.83 | 2.82 | 2.15 | |

TABLE CS-2
Beneficial Role of Herbal Formulation on Fasting Blood Glucose Level
among Type 2 Diabetes Mellitus Cases

| | | | Fasting Blood Glucose Level | | | Comp. |
|---|---|---|---|---|---|---|
| | | No. | (mg/dL) | | | Initial vs |
| | | of | | 6 Months | 1 Year | After 1 year |
| Treatment | sex | Cases | Initial | Treatment | Treatment | treatment |
| Conventional | M | 61 | 148.45 ± | 121.91 ± | 116.75 ± | P < 0.001 |
| Drug Treated | | | 29.82 | 23.73 | 21.41 | |
| | F | 38 | 143.75 ± | 124.87 ± | 121.92 ± | P < 0.001 |
| | | | 20.84 | 18.66 | 20.43 | |
| Conventional | M | 72 | 183.75 ± | 117.34 ± | 110.45 ± | P < 0.001 |
| Drug + | | | 24.91 | 19.28 | 14.77 | |
| Herbal | F | 43 | 154.73 ± | 122.92 ± | 108.73 ± | P < 0.001 |
| Formulation | | | 23.47 | 21.03 | 16.85 | |

Normal range: 70-120 mg/dl

TABLE CS-3
Decrease in Post Prandial Blood Glucose Level Following Herbal
Formulation Treatment in Type 2 Diabetes Mellitus Cases

| | | | Post Prandial Blood Glucose | | | Comp. |
|---|---|---|---|---|---|---|
| | | No. | Level (mg/dL) | | | Initial vs |
| | | of | | 6 Months | 1 Year | After 1 year |
| Treatment | | Cases | Initial | Treatment | Treatment | treatment |
| Conventional | M | 61 | 314.91 ± | 188.72 ± | 169.42 ± | P < 0.001 |
| Drug Treated | | | 41.68 | 30.91 | 33.71 | |
| | F | 38 | 331.83 ± | 204.87 ± | 178.91 ± | P < 0.001 |
| | | | 45.62 | 40.11 | 38.92 | |
| Conventional | M | 72 | 329.75 ± | 178.45 ± | 158.74 ± | P < 0.001 |
| Drug + | | | 44.90 | 33.77 | 40.82 | |
| Herbal | F | 43 | 334.12 ± | 182.96 ± | 164.91 ± | P < 0.001 |
| Formulation | | | 32.64 | 30.11 | 29.42 | |

Normal range: <140 mg/dl

TABLE CS-4
Decrease in Interleukin 6 Following Herbal Formulation Treatment in
Type 2 Diabetes Mellitus Cases

| | | | | | | Comp. |
|---|---|---|---|---|---|---|
| | | No. | Interleukin 6 (pg/ml) | | | Initial vs |
| | | of | | 6 Months | 1 Year | After 1 year |
| Treatment | | Cases | Initial | Treatment | Treatment | treatment |
| Conventional | M | 61 | 1.34 ± | 1.17 ± | 1.14 ± | P < 0.001 |
| Drug Treated | | | 0.20 | 0.18 | 0.21 | |
| | F | 38 | 1.08 ± | 1.29 ± | 1.22 ± | P < 0.05 |
| | | | 0.24 | 0.18 | 0.31 | |
| Conventional | M | 72 | 1.42 ± | 1.13 ± | 0.87 ± | P < 0.001 |
| Drug + | | | 0.23 | 0.34 | 0.22 | |
| Herbal | F | 43 | 1.34 ± | 0.96 ± | 0.81 ± | P < 0.001 |
| Formulation | | | 0.41 | 0.37 | 0.22 | |

Normal range: <1 pg/ml

TABLE CS-5
Effect of Herbal Formulation on CRP Level in Type 2 Diabetes
Mellitus Cases

| | | | | | | Comp. |
|---|---|---|---|---|---|---|
| | | No. | CRP Level (mg/L) | | | Initial vs |
| | | of | | 6 Months | 1 Year | After 1 year |
| Treatment | | Cases | Initial | Treatment | Treatment | treatment |
| Conventional | M | 61 | 2.84 ± | 2.91 ± | 2.58 ± | P < 0.05 |
| Drug Treated | | | 0.70 | 0.65 | 0.66 | |
| | F | 38 | 3.11 ± | 2.97 ± | 2.93 ± | P > 0.05 |
| | | | 0.45 | 0.34 | 0.39 | |
| Conventional | M | 72 | 3.21 ± | 2.58 ± | 2.13 ± | P < 0.001 |
| Drug + | | | 0.42 | 0.39 | 0.29 | |
| Herbal | F | 43 | 2.93 ± | 2.18 ± | 1.73 ± | P < 0.001 |
| Formulation | | | 0.42 | 0.41 | 0.32 | |

Normal range: 1-3 mg/L

TABLE CS-6
Decrease in Inflammatory Biomarker TNF□ Following Herbal
Formulation Treatment in Type 2 Diabetes Mellitus Cases

| | | | | | | Comp. |
|---|---|---|---|---|---|---|
| | | No. | TNF-□ (pg/mL) | | | Initial vs |
| | | of | | 6 Months | 1 Year | After 1 year |
| Treatment | | Cases | Initial | Treatment | Treatment | treatment |
| Conventional | M | 61 | 162.41 ± | 157.39 ± | 159.75 ± | P > 0.05 |
| Drug Treated | | | 30.13 | 38.44 | 33.91 | |
| | F | 38 | 171.20 ± | 168.96 ± | 173.75 ± | P > 0.05 |
| | | | 25.73 | 31.04 | 35.08 | |
| Conventional | M | 72 | 168.97 ± | 134.73 ± | 121.91 ± | P < 0.001 |
| Drug + | | | 31.64 | 25.13 | 18.99 | |
| Herbal | F | 43 | 182.73 ± | 148.96 ± | 136.94 ± | P < 0.001 |
| Formulation | | | 38.11 | 29.75 | 23.84 | |

Normal range: 25-800 pg/ml

TABLE CS-7
Beneficial Role of the Herbal Formulation on Endotheline Level among
Type 2 Diabetes Mellitus Cases

| | | | | | | Comp. |
|---|---|---|---|---|---|---|
| | | No. | Endotheline (pg/mL) | | | Initial vs |
| | | of | | 6 Months | 1 Year | After 1 year |
| Treatment | | Cases | Initial | Treatment | Treatment | treatment |
| Conventional | M | 61 | 613.91 ± | 590.84 ± | 578.94 ± | P < 0.01 |
| Drug Treated | | | 63.54 | 71.90 | 73.84 | |
| | F | 38 | 438.75 ± | 416.22 ± | 387.81 ± | P < 0.001 |
| | | | 41.97 | 53.17 | 58.27 | |
| Conventional | M | 72 | 578.89 ± | 409.64 ± | 373.91 ± | P < 0.001 |
| Drug + | | | 64.34 | 52.14 | 35.87 | |
| Herbal | F | 43 | 641.93 ± | 538.87 ± | 413.92 ± | P < 0.001 |
| Formulation | | | 51.34 | 48.90 | 58.25 | |

Normal range: 0.32-1000 pg/ml

TABLE CS-8
Decrease in Apolipoprotein B Following Herbal Formulation
Treatment in Type 2 Diabetes Mellitus Cases

| | | | | | | Comp. |
|---|---|---|---|---|---|---|
| | | No. | Apolipoprotein B (mg/dL) | | | Initial vs |
| | | of | | 6 Months | 1 Year | After 1 year |
| Treatment | | Cases | Initial | Treatment | Treatment | treatment |
| Conventional | M | 61 | 145.97 ± | 151.39 ± | 148.92 ± | P > 0.05 |
| Drug Treated | | | 30.21 | 25.97 | 26.82 | |
| | F | 38 | 161.31 ± | 173.14 ± | 164.45 ± | P > 0.05 |
| | | | 25.75 | 28.72 | 21.87 | |
| Conventional | M | 72 | 168.99 ± | 145.90 ± | 132.78 ± | P < 0.001 |
| Drug + | | | 32.42 | 25.82 | 30.01 | |
| Herbal | F | 43 | 158.72 ± | 124.84 ± | 116.72 ± | P < 0.001 |
| Formulation | | | 27.08 | 24.88 | 17.91 | |

Normal range: 55-159 mg/dl

TABLE CS-9
Beneficial Role of the Herbal Formulation in Decreasing the
Homocysteine Level among Type 2 Diabetes Mellitus Cases

| | | | | | | Comp. |
|---|---|---|---|---|---|---|
| | | No. | Homocysteine (mmol/L) | | | Initial vs |
| | | of | | 6 Months | 1 Year | After 1 year |
| Treatment | | Cases | Initial | Treatment | Treatment | treatment |
| Conventional | M | 61 | 38.79 ± | 37.24 ± | 37.91 ± | P > 0.05 |
| Drug Treated | | | 4.11 | 3.89 | 4.24 | |
| | F | 38 | 35.82 ± | 36.14 ± | 36.82 ± | P > 0.05 |
| | | | 6.31 | 5.91 | 5.32 | |
| Conventional | M | 72 | 41.02 ± | 33.71 ± | 27.94 ± | P < 0.001 |
| Drug + | | | 4.92 | 4.06 | 3.75 | |
| Herbal | F | 43 | 37.85 ± | 31.04 ± | 26.42 ± | P < 0.001 |
| Formulation | | | 5.17 | 3.21 | 3.81 | |

Normal range: 5-15 mmol/L

TABLE CS-10
Decrease in Leptin level following test drug treatment in Type 2
Diabetes Mellitus Cases

| | | | | | | Comp. |
|---|---|---|---|---|---|---|
| | | No. | Leptin(μgm/L) | | | Initial vs |
| | | of | | 6 Months | 1 Year | After 1 year |
| Treatment | | Cases | Initial | Treatment | Treatment | treatment |
| Conventional | M | 61 | 23.74 ± | 21.92 ± | 20.85 ± | P < 0.001 |
| Drug Treated | | | 3.11 | 2.87 | 2.93 | |
| | F | 38 | 26.11 ± | 24.93 ± | 23.40 ± | P < 0.001 |
| | | | 4.01 | 4.11 | 3.05 | |
| Conventional | M | 72 | 25.83 ± | 20.75 ± | 14.82 ± | P < 0.001 |
| Drug + | | | 3.14 | 3.28 | 2.64 | |
| Herbal | F | 43 | 28.93 ± | 23.91 ± | 19.87 ± | P < 0.001 |
| Formulation | | | 3.21 | 2.94 | 3.01 | |

Normal range: 5-12 μgm/L

TABLE CS-11
Mmodulation of adiponectin following Herbal formulation treatment
in Type 2 Diabetes Mellitus Cases

| | | | | | | Comp. |
|---|---|---|---|---|---|---|
| | | No. | Adiponectin(μg/ml) | | | Initial vs |
| | | of | | 6 Months | 1 Year | After 1 year |
| Treatment | | Cases | Initial | Treatment | Treatment | treatment |
| Conventional | M | 61 | 16.88 ± | 17.82 ± | 17.20 ± | P > 0.05 |
| Drug Treated | | | 3.03 | 2.61 | 2.72 | |
| | F | 38 | 13.84 ± | 14.64 ± | 14.82 ± | P > 0.05 |
| | | | 2.71 | 3.11 | 2.84 | |
| Conventional | M | 72 | 15.35 ± | 18.11 ± | 23.91 ± | P < 0.001 |
| Drug + | | | 2.62 | 4.01 | 3.73 | |
| Herbal | F | 43 | 12.96 ± | 16.35 ± | 19.11 ± | P < 0.001 |
| Formulation | | | 3.09 | 2.87 | 2.93 | |

Normal range: 5-30 μg/ml

We claim:

1) An herbal formulation for the prevention and management of Type 2 diabetes and associated risk factors comprising:
an effective amount of a hydro-alcoholic extract of Salacia roxburghii and a hydro-alcoholic extract of Salacia oblonga.

2) The herbal formulation of claim 1, further comprising one or more of minerals, vitamins, salts, filler and binders.

3) The herbal formulation of claim 1, comprising

| | |
|---|---|
| Salacia roxburghii | 450-900 mg/day |
| Salacia oblonga | 400-900 mg/day. |

4) The herbal formulation of claim 1, wherein the extracts are hydro-alcoholic extracts of Salacia roxburghii root and Salacia oblonga root.

5) A method of managing Type 2 diabetes and associated CHD risk factors in a subject suffering from diabetes, comprising administering the herbal formulation of claim 1 to the subject.

6) The method of claim 5, wherein the formulation provides alpha glucosidase, amylase inhibitory properties and lipid lipase lowering effects in the subject.

7) The method of claim 5, wherein the extract of Salacia roxburghii acts as an agonist for peroxisome proliferaters activators (PPAR) activated receptor 7 and a, and regulates insulin responsiveness gene transcription involved in glucose production transport and utilization, thus reducing blood glucose level in the subject.

8) The method of claim 5, wherein the hydro-alcoholic extract of Salacia roxburghii has potent anti-diabetic role in the subject.

9) The method of claim 5, wherein the hydro-alcoholic extract of Salacia roxburghii and Salacia oblonga reduces elevated triglycerides along with blood glucose level in the subject.

10) The method of claim 5, wherein the formulation reduces Apolipo B and increases good cholesterol HDL-c in the subject.

11) The method of claim 5, wherein the formulation has anti-atherosclerotic property by reducing oxidized LDL-c in diabetes and improving endothelial dysfunction in the subject.

12) The method of claim 5, wherein the formulation reduces vascular inflammation by reducing IL-6, TNF-α and CRP in the subject.

13) The method of claim 5, wherein the the formulation has anti-atherosclerotic and anti-obesity effects in the subject.

14) The method of claim 5, wherein the formulation provides antioxidant potentials through homocysteine regulation in the subject.

15) A process for the preparation of an herbal formulation for the prevention and management of Type 2 diabetes and associated risk factors, comprising
preparing a hydro-alcoholic extract of Salacia roxburghii and a hydro-alcoholic extract Salacia oblonga by extracting the Salacia roxburghii and Salacia oblonga with aqueous ethanol in 30:70 ratio of water to ethanol at 70-80° C. while maintaining pH of solution between 7-10,
subjecting the hydro-alcoholic extracts to separation by chromatography, and
characterizing molecules in the extracts by using IR and NMR.

16) The method of claim 15, wherein the extracts are root extracts.
